(19) United States
(12) Patent Application Publication
(10) Pub. No.: US 2015/0359720 A1
(43) Pub. Date: Dec. 17, 2015

(54) COSMETIC COMPOSITIONS

(71) Applicants: Christine Mendrok-Edinger, Basel (CH); Karina Radomsky, Basel (CH)

(72) Inventors: Christine Mendrok-Edinger, Basel (CH); Karina Radomsky, Basel (CH)

(73) Assignee: DSM IP ASSETS B.V., Heerlen (NL)

(21) Appl. No.: 14/124,672

(22) PCT Filed: Jun. 6, 2012
(86) PCT No.: PCT/EP2012/060658

§ 371 Date: Apr. 23, 2014

(30) Foreign Application Priority Data
Jun. 8, 2011 (EP) 11169196.0
Jul. 12, 2011 (EP) 11173605.4

(51) Int. Cl. A61K 8/49; A61K 8/35; A61K 8/06; A61K 8/55; A61K 8/37; A61Q 17/04

(52) U.S. Cl. CPC A61K 8/496; A61K 8/37; A61Q 17/04; A61K 8/062; A61K 8/55; A61K 8/35; A61K 2800/52

(57) ABSTRACT

The present invention relates to a composition for topical application comprising at least a benzotriazol derivative, at least one dibenzoylmethane derivative and a cosmetic solvent selected from the group consisting of fatty acid triglycerides and/or sebacic acid esters or diesters. Furthermore, the invention relates to the use of at least one specific benzotriazol derivative for in creasing the water resistance of a dibenzoylmethane derivatives in a topical composition comprising a cosmetic solvent selected from the group consisting of fatty acid triglycerides and/or sebacic acid esters or diesters.

The present invention relates to a composition for topical application comprising at least a benzotriazol derivative, at least one dibenzoylmethane derivative and a cosmetic solvent selected from the group consisting of fatty acid triglycerides and/or sebacic acid esters or diesters. Furthermore, the invention relates to the use of at least one specific benzotriazol derivative for increasing the water resistance of a dibenzoylmethane derivatives in a topical composition comprising a cosmetic solvent selected from the group consisting of fatty acid triglycerides and/or sebacic acid esters or diesters.

Sun care products have evolved considerably over the years. Earlier formulations were intended to protect the user from UV-B radiation as was once thought that UV-B rays were the most important contributors to wrinkling, skin disease, and skin cancer. However, more recent studies have shown that UV-A radiation is equally or even more important in the development of solar damage and skin diseases, such as lupus erythematosus and melanoma and non-melanoma skin cancer. Thus, today's focus is towards eliminating as much of UVA (320-400 nm) and/or UVB (280-320 nm) light as possible. Consequently, there's a constantly increasing need for sun care products exhibiting high SPF's (Sun Protection Factor) and high UVA protection while being photostable.

Water resistance of sun care products is an ongoing issue and methods of increasing the water resistance of sun care product respectively individual UV-filter substances such as e.g. the most commonly used UV-A filter substance (INCI names) butyl methoxy dibenzoylmethane are highly desirably. Furthermore, sun care products are often sticky which e.g. leads to an increased sand affinity to the skin which is highly unwanted.

It was therefore the object of the present invention to remedy the disadvantages of the prior art and to develop sun care comprising dibenzoylmethane derivatives which exhibit an improved water resistance and thus UV-protection even after bathing. Furthermore, such compositions should favourably also exhibit a reduced stickiness resulting in a reduced affinity of sand to skin.

Surprisingly it has been found that the water resistance of dibenzoylmethane derivatives such as butyl methoxydibenzoylmethane in sun care products comprising a cosmetic solvent selected from the group consisting of fatty acid triglycerides and/or sebacic acid esters or diesters can be significantly improved by the addition of at least one specific benzotriazole derivative. Furthermore, the stickiness of the compositions was reduced resulting in a reduced sand adherence.

Thus, the invention relates to a topical composition comprising a cosmetic solvent selected from the group consisting of fatty acid triglycerides and/or sebacic acid esters or diesters, at least one dibenzoylmethane derivative and at least one benzotriazol derivative of formula (I)

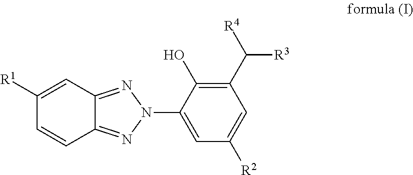

wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl; preferably hydrogen.

Furthermore, the invention relates to the use of at least one benzotriazol derivative of formula (I) for increasing the water resistance of at least one dibenzoylmethane derivative in a topical composition comprising a cosmetic solvent selected from the group consisting of fatty acid triglycerides and/or sebacic acid esters or diesters.

In a further aspect the invention relates to a method for increasing the water resistance of a dibenzoylmethane derivative in a topical composition comprising a cosmetic solvent selected from the group consisting of fatty acid triglycerides and/or sebacic acid esters or diesters, said method comprising the addition of at least one benzotriazol derivative of formula (I) into said topical composition and observing or appreciating the result.

Another subject matter of the invention is directed to a method for reducing the sand adherence on skin after application of a topical composition comprising a cosmetic solvent selected from the group consisting of fatty acid triglycerides and/or sebacic acid esters or diesters, said method comprising the addition of at least one benzotriazol derivative of formula (I) into said topical composition.

The term “C_{x-y}alkyl” as used herein refers to straight-chain or branched alkyl radicals having x to y carbon atoms such as e.g. methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-di methyl propyl, 1-methylpentyl, 2-methyl pentyl, 3-methylpentyl, 4-methylpentyl, 1,1-di methyl propyl, 1,2-dimethylbutyl, 1,3-di methylbutyl, 2,2-di methylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl without being limited thereto.

The term C₅₋₁₀cycloalkyl denotes to unsubstituted or C₁₋₁₀alkyl (mono- or poly-)substituted, such as in particular C₁₋₅alkyl (mono- or poly-)substituted cyclic, bicyclic or tricyclic hydrocarbon residues (e.g. unsubstituted or C₁₋₁₀alkyl (mono- or poly-)substituted cyclopentyl, cyclohexyl, cycoheptyl or decahydronaphtyl residues). Preferably, C₅₋₁₀cycloalkyl denotes to unsubstituted or C₁₋₂alkyl (mono- or poly-)substituted cyclopentyl, cyclohexyl or cycoheptyl residues such as in particular to unsubstituted or (mono- or poly-) methyl substituted cyclohexyl such as most in particular to cyclohexyl or 3,3,5-trimethyl-cyclohexyl.

The term “C_{x-y}alkoxy” as used herein denotes to linear or branched alkoxy-, respectively unsubstituted or (mono- or poly-)substituted cycloalkoxy radicals having from x to y carbon atoms such as e.g. methoxy, ethoxy, propoxy, butyloxy or pentyloxy, 2,5,5-trimethylhexyloxy, 3,5,5-trimethylhexyloxy, isoamyloxy, 2-ethylhexyloxy or 3,3,5-trimethyl-cyclohexyloxy.

The term C₆₋₁₀aryl refers e.g. to naphthyl or phenyl radicals, preferably phenyl.

The term “cosmetic solvent” as used herein relates to any solvent conventionally used in topical, in particular topical cosmetic compositions. The cosmetic solvents have the functional use to dissolve other ingredients such as in particular solid ingredients, e.g. solid UV-filter substances such as e.g. butyl methoxy dibenzoylmethane. Furthermore the cosmetic solvents are liquid at ambient temperature (25° C.).

Particular suitable dibenzoylmethane derivatives according to the invention encompass 2-methyldibenzoylmethane, 4-methyldibenzoylmethane, 4-isopropyldibenzoylmethane, 4-tert.-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 4-tert.-butyl-4′-methoxydibenzoylmethane, 2-methyl-5-isopropyl-4′-methoxydibenzoylmethane, 2-methyl-5-tert-butyl-4′-methxydibenzoylmethane, 2,4-dimethyl-4′-methoxydibenzoylmethane, 2,6-dimethyl-4-tert-butyl-4′-methoxydibenzoylmethane such as most in particular 4-(tert.-butyl)-4′-methoxydibenzoylmethane (INCI Name: butyl methoxy dibenzoylmethane; IUPAC Name: 1-(4-MethoxyphenyI)-3-(4-tert-butylphenyl)propane-1,3-dione) which is e.g. commercially available as PARSOL® 1789 at DSM Nutritional Products Ltd Kaiseraugst.

The amount of the dibenzoylmethane derivative such as in particular of butyl methoxy dibenzoylmethane in the compositions according to the invention is preferable selected in the range of 2 to 8 wt.-%, in particular in the range of 3 to 6 wt.-%, most particular in the range of 3 to 5 wt.-% based on the total weight of the composition.

The amount of the at least one benzotriazol derivative of formula (I) in the compositions according to the invention is at least 0.01 wt.-%. In particular, the amount of the at least one benzotriazol derivative of formula (I) in the compositions according to the invention is selected in the range of 1 to 20 wt.-%, such as in the range of 2 to 20 wt.-%, and preferably in the range of 2 to 15 wt.-%, more preferably in the range of 4 to 12 wt.-%, even more preferably in the range of 6 to 12 wt.-%, and most preferably in the range of 6 to 10 wt.-%, based on the total weight of the composition.

In a particular embodiment of the present invention the benzotriazol derivative is selected from compounds of formula (I) wherein R¹ and R⁴ are hydrogen, R² is methyl and R³ is C₆₋₁₀alkoxy such as preferably C₆₋₁₀alkoxy, or C₆cycloalkoxy such as in particular 2,5,5-trimethylhexyloxy, 3,5,5-trimethylhexyloxy, isoamyloxy, 2-ethylhexyloxy or 3,3,5-trimethyl-cyclohexyloxy. Such compounds and their preparation are e.g. disclosed in EP Application No.: 10150832.3 (PCT publication: WO2011/086124). Particularly preferred according to the present invention is a compound of formula (I), wherein R¹ and R⁴ are hydrogen, R² is methyl and R³ is 2-ethylhexyloxy (i.e. 2-(2H-Benzotriazol-2-yl)-6-(2-ethylhexyloxymethyl)-4-methyl-phenol).

In another particular embodiment of the present invention the compound of formula (I) is a compound wherein R¹ and R⁴ are hydrogen, R² is methyl and R³ is undecyl (C₁₁H₂₃) which is commercially available as Tinogard TL [INCI Name: benzotriazolyl dodecyl p-cresol; IUPAC Name: 2-(2H-benzotriazol-2-yl)-6-dodecyl-4-methyl-phenol] at BASF SE Ludwigshafen.

Suitable sebacic acid ester or diesters encompass esters of sebacic acid with straight-chain or branched alkanols, alkandiols and/or alkantriols having from 1 to 36 carbon atoms or polyols such as glycerin or diglycerin. Exemplary sebacic acid ester or diesters encompass e.g. Dibutyloctyl Sebacate, Dibutyl Sebacate, Dicaprylyl/Capryl Sebacate, Diethylhexyl sebacate, Diethyl Sebacate, Diglyceryl Sebacate/Isopalmitate (Polyglyceryl-2 Isopalmitate/Sebacate is a mixed ester of Isopalmitic Acid, Sebacic Acid and Diglycerin), Dihexyldecyl Sebacate, Diisooctyl Sebacate, Diisostearyl Sebacate, Dioctyldodecyl Sebacate, Ditrimethylolpropane Isostearate/Sebacate (Di-2,2-dihydroxymethylbutyl ether, tetraesters with isooctadecanoic acid and decanedioic acid), Isostearyl Sebacate without being limited thereto. Particularly preferred is Diisopropyl sebacate (commercially available as DUB DIS at Stearinerie Dubois Fils, Boulogne Billancourt Cedex).

Suitable fatty acid triglycerides encompass in particular triglycerides of straight chain or branched, saturated or unsaturated C6-36 fatty acids, such as particularly C6-18 fatty acids, which optionally may be substituted with one or several, preferably one or two, hydroxy groups. Exemplary fatty acid triglycerides encompass for example Caprylic/Capric Triglyceride, Lauric/Palmitic/Oleic Triglyceride, Caprylic/Capric/Lauric Triglyceride, Caprylic/Capric/Linoleic Triglyceride, Caprylic/Capric/Myristic/Stearic Triglyceride, Caprylic/Capric/Palmitic/Stearic Triglyceride, Caprylic/Capric/Stearic Triglyceride, Caprylic/Capric/Succinic Triglyceride, Lauric/Palmitic/Oleic Triglyceride, Mustelic/Palmitic Triglyceride, Oleic/Linoleic Triglyceride, Oleic/Palmitic/Lauric/Myristic/Linoleic Triglyceride, Palmitic/Stearic Triglyceride, and Ricinoleic/Caproic/Caprylic/Capric Triglyceride. Preferably, the fatty acid triglycerides are selected from triglycerides of straight chain or branched C6-18 fatty acids such as particularly C6-18 fatty acids. Most in particular the fatty acid triglycerides is Caprylic/Capric Triglyceride (Myritol® 318 from Cognis).

The amount of the sebacic acid ester or diester such as in particular of diisopropyl sebacate in the compositions according to the invention is preferable selected in the range of 1 to 50 wt.-%, in particular in the range of 5 to 30 wt.-%, such as about 10 to 30 wt.-%, based on the total weight of the composition.

The amount of the fatty acid triglyceride such as in particular of caprylic/capric triglyceride in the compositions according to the invention is preferable selected in the range of 1 to 50 wt.-%, in particular in the range of 5 to 40 wt.-%, such as about 10-30 wt.-%, based on the total weight of the composition.

In a particular embodiment according to the present invention, the topical compositions according to the present invention comprise besides the cosmetic solvents selected from the group consisting of fatty acid triglycerides and/or sebacic acid esters or diesters no further cosmetic solvents such as particularly no Finsolv TN (i.e. C12-C16 alkyl benzoate).

In a further particular embodiment the compositions according to the present invention further comprise homomenthyl salicylate (e.g. commercially available as PARSOL HMS at DSM Nutritional Products Ltd Kaiseraugst). The homomenthyl salicylate is preferably used in an amount ranging from 1 to 25 wt.-% such as in an amount of 5 to 15 wt.-%, preferably in the range of 6 to 10 wt.-%, such as about 10 wt.-%, based on the total weight of the composition.

In another particular embodiment the compositions according to the present invention are substantially free of a polyglycerol based UV-filter such as e.g. disclosed in [EP Application No's] EP09178503.0, EP09178501.4, EP09178502.2 EP09178495.9, EP09178506.3, EP09178505.5 or EP10150832.3 which are obtainable by a process comprising the steps of ring-opening polymerization of x mol equivalents of glycidol using 1 mol equivalent of a polyol starter unit with y mol equivalents hydroxyl-groups, followed by block copolymerization with z×(x+y) mole equivalents of propylene oxide to form a hyperbranched polyether-polyol backbone carrying (x+y) mol equivalents hydroxyl-groups followed by partial or total esterification, respectively partial or total etherification of the hydroxyl groups with a UV-light absorbing chromophore such as particularly with p-dimethylamino benzoic acid, 3-[1-(4-Hydroxymethyl-phenyl)-meth-(E)-ylidene]-1,7,7-trimethyl-bicyclo[2.2.1]heptan-2-one, 2-(4-diethylamino-2-hydroxybenzoyl)benzoic acid, p-alkoxycinnamic acid, 2-cyano-3,3-diphenylacrylic acid as well as mixtures thereof and wherein x is an integer selected in the range from 3-16, y is an integer selected in the range from 1-6, and z is an integer selected in the range from 0-10.

The term “topical” is understood here to mean external application to keratinous substances, which are in particular the skin, scalp, eyelashes, eyebrows, nails, mucous membranes and hair.

As the compositions according to the invention are intended for topical application, they comprise a physiologically acceptable medium, that is to say a medium compatible with keratinous substances, such as the skin, mucous membranes, and keratinous fibres. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are light-protective preparations (sun care products), such as sun protection milks, sun protection lotions, sun protection creams, sun protection oils, sun blocks or tropical's or day care creams with a SPF (sun protection factor). Of particular interest are sun protection creams, sun protection lotions, sun protection milks and sun protection preparations.

The topical compositions according to the present invention may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si-)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion or other usual forms, which can also be applied by pens, as masks or as sprays.

The topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art and illustrated in the examples.

If the topical composition according to the invention is an O/W emulsion, then it contains advantageously at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self emulsifying), stearic acid, salts of stearic acid, polyglyceryl-3-methylglycosedistearate. Further suitable emulsifiers are phosphate esters and the salts thereof such as cetyl phosphate (Amphisol® A), diethanolamine cetyl phosphate (Amphisol®DEA), potassium cetyl phosphate (Amphisol® K), sodiumcetearylsulfat, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

The at least one 0/W respectively Si/W emulsifier is preferably used in an amount of 0.5 to 10 wt.-% such as in particular in the range of 0.5 to 5 wt.-% such as most in particular in the range of 1 to 4 wt.-% based on the total weight of the composition.

Particular suitable O/W emulsifiers according to the present invention encompass phosphate ester emulsifiers of formula (II)

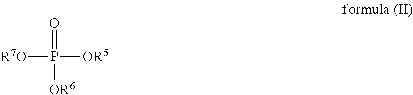

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular advantageous phosphate ester emulsifier according to the invention is potassium cetyl phosphate e.g. commercially available as Amphisol® K at DSM Nutritional Products Ltd Kaiseraugst as the overall staining tendency of the benzotriazol derivative is decreased.

In particular embodiment, the topical compositions according to the present invention are in the form of O/W emulsions comprising an oily phase dispersed in an aqueous phase in the presence of an O/W emulsifier, wherein the benzotriazol derivative of formula (I) is benzotriazolyl dodecyl p-cresol, the cosmetic solvent is diisopropyl sebacate and/or caprylic/capric triglyceride, and the O/W emulsifier is potassium cetyl phosphate.

In another particular embodiment, the topical compositions according to the present invention are in the form of O/W emulsions comprising an oily phase dispersed in an aqueous phase in the presence of an O/W emulsifier wherein the benzotriazol derivative of formula (I) is a compound of formula (I) wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy, the cosmetic solvent is diisopropyl sebacate and/or caprylic/capric triglyceride, and the O/W emulsifier is potassium cetyl phosphate.

The topical compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 5 wt.-%, such as most in particular in the range of 1 to 3 wt.-%, based on the total weight of the composition. Particular suitable co-surfactants are selected from the list of alkyl alcohols such as cetyl alcohol (Lorol C16, Lanette 16) cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof. In the case the O/W emulsifier is a phosphate ester emulsifier such as particularly potassium cetyl phosphate a particular preferred co-surfactant is cetearyl alcohol (Lanette O)

The compositions in form of O/W emulsions according to the invention can be provided, for example, in all the formulation forms for O/W emulsions, for example in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

Finally, a subject-matter of the invention is a method for the cosmetic treatment of keratinous substances such as in particular the skin, characterized in that a composition as defined above is applied to the said keratinous substances such as in particular to the skin. The method is in particular suitable to protect the skin against the adverse effects of UV-radiation such as in particular sun-burn and/or photoageing.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the CTFA Cosmetic Ingredient Handbook, Second Edition (1992), The Cosmetic, Toiletry and Fragrance Association, Inc. or International Cosmetic Ingredient Dictionary and Handbook, Vol. 4, 2008 (ISBN-10: 1882621433) without being limited thereto.

The necessary amounts of the cosmetic and dermatological adjuvants and additives can based on the desired product easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7. The pH can easily be adjusted as desired with suitable acids such as e.g. citric acid or bases such as NaOH, Triethanolamine (TEA Care), Tromethamine (Trizma Base) and Aminomethyl Propanol (AMP-Ultra PC 2000) according to standard methods in the art.

The following examples are provided to further illustrate the compositions and effects of the present invention. These examples are illustrative only and are not intended to limit the scope of the invention in any way.

### EXAMPLE 1

### Water Resistance

0.75 mg/cm² of the respective compositions as outlined in table 1 and 2 were applied to PMMA plates and the plates were dried at RT for 30 min. Afterwards the initial in vitro SPF (Sun Protection Factor) were determined with a “m·u·t HELIAS fiber optical integrator”. Then the plates were immersed into a flask filled with 2.5 l water (bi-distilled) for 20 min while the water was stirred with a magnetic stirrer at 450 1/min at RT (The plates were attached at the edge of the flask with a clothespin, such that the side covered with the composition was directed into the flask). Afterwards the plates were dried at 40° C. for 30 min. The immersion/drying procedure was repeated once. After final drying the in-vitro SPF was measured again and the water resistance was calculated as % WR=[(SPF after immersion−1)/(SPF initial−1)]*100 and the Δ water resistance as (% WR of sample with benzotriazol derivative)−(% WR of Sample without benzotriazol derivative)

Each composition was tested separately on 4 plates.

The water resistance for each composition was determined as mean value from the 4 plates.

Ref1=Respective reference example without benzotriazol derivative
Ref2=Respective reference example with benzotriazol derivative and other UV-filter
Inv=Example according to the invention

TABLE 1
Formulations with caprylic/capric triglyceride (Myritol ® 318)

| | Ref1 | Inv | Inv | Ref1 | Ref2 | Ref2 | Ref 1 | Ref2 |
|---|---|---|---|---|---|---|---|---|
| Tradename | wt.-% | | | wt.-% | | | wt.-% | |
| Myritol ® 318 | 25 | 25 | 25 | 25 | 25 | 25 | 25 | 25 |
| Parso ®I HMS | 10 | 10 | 10 | 10 | 10 | 10 | 10 | 10 |
| Tinogard ® TL | | 10 | | | 10 | | | 10 |
| 2-(2H-Benzo- | | 10 | | | 10 | | | |
| triazol-2-yl)-6- | | | | | | | | |
| (2-ethylhexyl- | | | | | | | | |
| oxy-methyl)-4- | | | | | | | | |
| methyl-phenol* | | | | | | | | |
| Parsol ® 1789 | 4 | 4 | 4 | | | | | |
| Uvinul ® T150 | | | | 4 | 4 | 4 | | |
| Parsol ® MCX | | | | | | | 10 | 10 |
| Amphisol ® K | 2.5 | 2.5 | 2.5 | 2.5 | 2.5 | 2.5 | 2.5 | 2.5 |
| Lanette ® O | 1.5 | 1.5 | 1.5 | 1.5 | 1.5 | 1.5 | 1.5 | 1.5 |
| Phenonip ® | 0.8 | 0.8 | 0.8 | 0.8 | 0.8 | 0.8 | 0.8 | 0.8 |
| Keltrol ® | 0.3 | 0.3 | 0.3 | 0.3 | 0.3 | 0.3 | 0.3 | 0.3 |
| CG-T | | | | | | | | |
| Water | Ad 100 | | | Ad 100 | | | Ad 100 | |
| in vitro SPF | 7.4 | 29.6 | 19.1 | 6.3 | 35.4 | 21.0 | 9.4 | 43.7 |
| initial | | | | | | | | |
| in vitro SPF | 3.6 | 17.7 | 9.4 | 4.6 | 19.2 | 10.2 | 6.9 | 27.3 |
| after immersion | | | | | | | | |
| Water | 41% | 58% | 46% | 68% | 53% | 46% | 70% | 62% |
| Resistance | | | | | | | | |
| Δ water | | +17% | +5% | | −15% | −22% | | −8% |
| Resistance | | | | | | | | |

*benzotriazol derivative of formula (I): 2-(2H-Benzotriazol-2-yl)-6-(2-ethylhexyloxymethyl)-4-methyl-phenol (i.e. a compound of formula (I), wherein R¹ and R⁴ are hydrogen, R² is methyl and R³ is 2-ethylhexyloxy)

TABLE 2
Formulations with diisopryl sebacate (DUB DIS)

| | Ref1 | Inv | Ref1 | Ref2 | Ref1 | Ref2 |
|---|---|---|---|---|---|---|
| Tradename | wt.-% | | wt.-% | | wt.-% | |
| DUB DIS | 25 | 25 | 25 | 25 | 25 | 25 |
| Parsol ® HMS | 10 | 10 | 10 | 10 | 10 | 10 |
| Tinogard ® TL | | 10 | | 10 | | 10 |
| Parsol ® 1789 | 4 | 4 | | | | |
| Uvinul ® T150 | | | 4 | 4 | | |
| Parsol ® MCX | | | | | 10 | 10 |
| Amphisol ® K | 2.5 | 2.5 | 2.5 | 2.5 | 2.5 | 2.5 |
| Lanette ® O | 1.5 | 1.5 | 1.5 | 1.5 | 1.5 | 1.5 |
| Phenonip ® | 0.8 | 0.8 | 0.8 | 0.8 | 0.8 | 0.8 |
| Keltrol ® CG-T | 0.3 | 0.3 | 0.3 | 0.3 | 0.3 | 0.3 |
| Water | Ad 100 | | Ad 100 | | Ad 100 | |
| in vitro SPF initial | 7.3 | 24.4 | 6.2 | 27.7 | 10.5 | 36 |
| in vitro SPF after | 3.7 | 15.2 | 4.7 | 15 | 8.3 | 25.5 |
| immersion | | | | | | |
| Water Resistance | 43% | 61% | 71% | 52% | 77% | 70% |
| Δ Water resistance | | +18 | | −19% | | −7% |

As can be retrieved from the table the addition of a benzotriazol derivative according to the present invention significantly increases the water resistance of the dibenzoylmethane derivative whereas the water resistance of the other UV filters is decreased.

TABLE 3
Formulations with C₁₂₋₁₅ alkyl benzoate (Finsolv TN) (Reference)

| Tradename | wt.-% | | |
|---|---|---|---|
| Finsolv TN | 25 | 25 | 25 |
| Parsol ® HMS | 10 | 10 | 10 |
| Tinogard ® TL | — | 10 | — |
| 2-(2H-Benzo-triazol-2-yl)-6-(2-ethylhexyl-oxy- | — | — | 10 |
| methyl)-4-methyl-phenol* | | | |
| Parsol ® 1789 | 4 | 4 | 4 |
| Amphisol ® K | 2.5 | 2.5 | 2.5 |
| Lanette ® O | 1.5 | 1.5 | 1.5 |
| Phenonip ® | 0.8 | 0.8 | 0.8 |
| Keltrol ® CG-T | 0.3 | 0.3 | 0.3 |
| Water | Ad 100 | | |
| in vitro SPF initial | 7.7 | 25 | 15.8 |
| in vitro SPF after immersion | 5.7 | 12.2 | 8.8 |
| Water Resistance | 70% | 47% | 53% |
| Δ Water resistance | | −23% | −17% |

As can be retrieved from table 3, no increased water resistance is observed in the presence of another solvent such as C₁₂₋₁₅ alkyl benzoate.

### EXAMPLE 2

### Sand Repellent Properties

50 mg of the respective formulation as outlined in table 2 were applied on PMMA Schonberg plates (5×5 cm). Plates are dried at 43° C. for 15 minutes. 2.5 g sand were distributed on the plates. For removal of excess sand the plates were tilt (about 45°, left and right side). The sticky sand residue on each plate was determined through weighting. The results are presented in table 2.

The reduction in the sand adherence was determined as:

Δ Adherence [%]={(Adherence Sample [mg]−Adherence Reference [mg])/Adherence Sample [mg]}*100%.

TABLE 4

| | | | |
|---|---|---|---|
| 5A | | 6A | |
| (Reference) | 5B | (Reference) | 6B |

| | | |
|---|---|---|
| Trade Name | wt.-% | wt.-% |

| | | | | |
|---|---|---|---|---|
| Amphisol ® K | 2.5 | 2.5 | 2.5 | 2.5 |
| Lanette ® O | 1.5 | 1.5 | 1.5 | 1.5 |
| Myritol ® 318 | 30 | 30 | — | — |
| DUB DIS | | | 30 | 30 |
| Phenonip ® | 0.8 | 0.8 | 0.8 | 0.8 |
| Keltrol ® CG-T | 0.3 | 0.3 | 0.3 | 0.3 |
| PARSOL ® 1789 | 4.00 | 4.00 | 4.00 | 4.00 |
| Tinogard ® TL | | 4.00 | | 4.00 |

| Water | Ad 100 | | Ad 100 | |
|---|---|---|---|---|
| Sand adherence [mg] | 1.30 | 1.23 | 1.21 | 1.11 |

| | | |
|---|---|---|
| Δ Adherence [%] | −5% | −8% |

1. A topical composition comprising a cosmetic solvent selected from the group consisting of fatty acid triglycerides and/or sebacic acid esters or diesters, at least one dibenzoylmethane derivative and at least one benzotriazol derivative of formula (I) 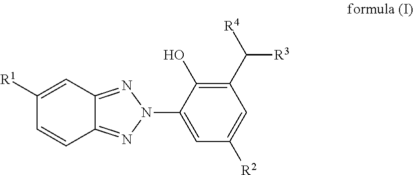
wherein
R¹ is hydrogen; C₁₋₅alkyl; C₁₋₅alkoxy or halogen; preferably hydrogen or chloride, most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl, most preferably methyl;
R³ is C₁₋₂₀alkyl, C₅₋₁₀cycloalkyl, C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl, preferably hydrogen.

2. The topical composition according to claim 1, characterized in that the benzotriazol derivative of formula (I) is used in an amount selected in the range of 1 to 20 wt.-% based on the total weight of the composition.

3. The topical composition according to claim 1, characterized in that the benzotriazol derivative is used in an amount selected in the range of 2 to 20 wt.-% based on the total weight of the composition.

4. The topical composition according to claim 1, characterized in that the benzotriazol compound of formula (I) is a compound wherein R¹ and R⁴ are hydrogen, R² is methyl and R³ is 2,5,5-trimethylhexyloxy, 3,5,5-trimethylhexyloxy, isoamyloxy, 2-ethylhexyloxy or 3,3,5-trimethyl-cyclohexyloxy or undecyl.

5. The topical composition according to claim 1, characterized in that the amount of the dibenzoylmethane derivative is selected in the range of 2 to 8 wt.-% based on the total weight of the composition.

6. The topical composition according to claim 1, characterized in that the cosmetic solvent is selected from diisopropyl sebacate and/or caprylic/capric triglyceride.

7. The topical composition according to claim 1, characterized in that the amount of the cosmetic solvent is selected in the range of 1 to 50 wt.-%, based on the total weight of the composition.

8. The topical composition according to claim 1, characterized in that the topical composition is an O/W emulsion comprising an oily phase dispersed in an aqueous phase in the presence of an O/W emulsifier.

9. The topical composition according to claim 8, characterized in that the O/W emulsifier is selected from the group of phosphate esters.

10. The topical composition according to claim 1, characterized in that the amount of O/W emulsifier is selected in the range of 0.5 to 10 wt.-%, based on the total weight of the composition.

11. The topical composition according to claim 1, characterized in that the composition comprises at least one co-surfactant in an amount selected in the range of 0.1 to 10 wt.-%, based on the total weight of the composition.

12. The topical composition according to claim 11, characterized in that the co-surfactant is selected from the group consisting of cetyl alcohol, cetearyl alcohol, stearyl alcohol, behenyl alcohol, glyceryl stearate, glyceryl myristate, hydrogenated coco-glycerides and mixtures thereof.

13. Use of at least one benzotriazol derivative of formula (I) 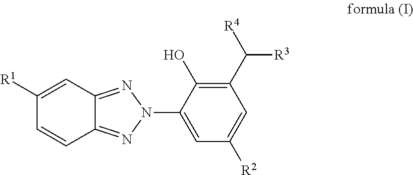
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl; C₅₋₁₀cycloalkyl; C₁₋₂₀alkoxy or C₅₋₁₀cycloalkoxy, preferably C₅₋₁₅alkyl or C₅₋₁₅alkoxy; and
R⁴ is hydrogen or C₁₋₅alkyl; preferably hydrogen for increasing the water resistance of a dibenzoylmethane derivative in a topical composition comprising a cosmetic solvent selected from the group consisting of fatty acid triglycerides and/or sebacic acid esters or diesters.

14. Method for increasing the water resistance of a dibenzoylmethane derivatives in a topical composition comprising a cosmetic solvent selected from the group consisting of fatty acid triglycerides and/or sebacic acid esters or diesters, said method comprising the addition of at least one benzotriazol derivative of formula (I) 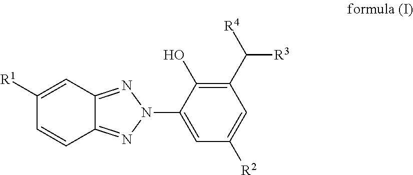
wherein
R¹ is hydrogen; C₁₋₅alkyl; C₁₋₅alkoxy or halogen; preferably hydrogen or chloride; most preferably hydrogen;
R² is hydrogen; C₁₋₂₀alkyl; C₁₋₅alkoxy; C₁₋₅alkoxycarbonyl; C₅₋₁₀cycloalkyl; C₆₋₁₀aryl or aralkyl; preferably hydrogen or C₁₋₅alkyl; most preferably methyl;
R³ is C₁₋₂₀alkyl, C₅₋₁₀cycloalkyl; C₁₋₂₀alkoxy or C₅₋₁₀cycloalkoxy; preferably C₅₋₁₅alkyl or C₅₋₁₅alkoxy; and
R⁴ is hydrogen or C₁₋₅alkyl; preferably hydrogen into said topical composition and observing or appreciating the result.

15. A method for the cosmetic treatment of keratinous substances, characterized in that a composition as defined in claim 1 is applied to the said keratinous substances.
